(19) United States
(12) Patent Application Publication
(10) Pub. No.: US 2009/0024190 A1
(43) Pub. Date: Jan. 22, 2009

(54) MEDICAL LASER DEVICE

(76) Inventor: Michael Irvine, Cambridge (GB)

Correspondence Address: AFS / ANGIODYNAMICS, 666 THIRD AVENUE, FLOOR 10, NEW YORK, NY, 10017, US

(21) Appl. No.: 12/280,506

(22) PCT Filed: Feb. 27, 2007
(86) PCT No.: PCT/GB2007/000682

§ 371 Date: Sep. 12, 2008

(30) Foreign Application Priority Data
Feb. 27, 2006 (GB) 0603866.5

(51) Int. Cl. A61N 5/06

(52) U.S. Cl. 607/89

(57) ABSTRACT

A medical laser device is disclosed comprising a laser source and an optical fibre which is inserted in use within an introducer sheath. A withdrawal assist device is provided to assist a user to withdraw the optical fibre and/or the introducer sheath at a controllable rate. The introducer sheath may have graduations provided on an outer surface of the sheath which are detected by the withdrawal-assist device.

The present invention relates to a medical laser device and a method, a withdrawal-assist device and a method of endovenous laser treatment. The preferred embodiment relates to a medical laser device comprising a withdrawal-assist device for assisting a user to manually withdraw an optical fibre and/or introducer sheath at a controllable, predetermined or desired rate.

In certain invasive medical procedures thermal or other energy may be administered to a patient with beneficial effects. For example, it is known to use energy to detect a tumour or a region of the body or to destroy or denature diseased or malfunctioning body tissue. U.S. Pat. No. 6,095,149, for example, describes a method of treatment of invertebral disc abnormalities with thermal energy. Other types of medical treatment utilise laser energy.

Laser energy may be delivered to an area of the body by means of an optical fibre such as a bare-tipped optical fibre. However, the tip of such optical fibres can cause trauma to soft tissues. Therefore, for some invasive applications, it is not desirable to insert the optical fibre directly into the body tissue. Instead, the optical fibre may instead be inserted into a flexible introducer sheath which acts to guide the optical fibre. The introducer sheath also protects the optical fibre (and the surrounding tissue) whilst it is being inserted into the region of the body to be treated. An introducer sheath may be inserted into body tissue over a guide wire which is then withdrawn. An optical fibre can then be inserted once the introducer sheath is in place in the body.

In use an introducer sheath may be positioned so that the optical fibre protrudes a few millimetres or centimetres beyond the end of the introducer sheath so as to deliver laser energy efficiently from the tip of the optical fibre to the surrounding tissue.

It is known to detect the protrusion of the optical fibre from the introducer sheath using direct visualisation of a red aiming laser beam from the optical fibre through the skin. However, visualisation of the laser beam is imprecise and at best is only a guide. Furthermore, visualisation is only possible in regions of the body which are sufficiently close to the surface of the skin.

The known technique is not therefore generally suitable for a number of different applications.

Since it can be difficult for an operator or surgeon using conventional arrangements to determine when the distal end of the optical fibre is approaching the distal end of the introducer sheath, damage can readily occur to soft tissue of a patient if the optical fibre is initially accidentally extended too far beyond the introducer sheath. In order to avoid causing an accidental injury to a patient an operator must insert the optical fibre very slowly into the introducer sheath and must proceed cautiously at all times thereby making the procedure relatively slow.

A specific type of cosmetic procedure known as Endovenous Laser Treatment (“EVLT”) (RTM) is known wherein laser energy is delivered to the inner wall of a vein in order to treat varicose veins. In use, laser radiation is delivered to the inner wall of a vein wherein it is absorbed, thermally re-structuring the vein.

A known medical laser device which may be used to perform such procedures is disclosed in WO 02/102266 (Diomed Inc.). The disclosed medical laser device comprises an optical fibre having markings which enable the optical fibre to be accurately positioned relative to an introducer sheath.

The markings allow an operator or surgeon to know when the distal end of the optical fibre is aligned with the end of the introducer sheath and also when the distal end of the optical fibre extends beyond the introducer sheath by a desired amount. The markings enable an operator or surgeon to quickly insert the optical fibre into the introducer sheath and enable the optical fibre to be positioned in the optimum position relative to the introducer sheath without risk of damage to either the optical fibre or the surrounding tissue.

The optical fibre may be secured to the introducer sheath at a position wherein a first marking is substantially in alignment with the proximal end of the introducer sheath. The optical fibre and the introducer sheath may then be positioned at a desired location using ultrasound. The introducer sheath can then be released from the optical fibre and the introducer sheath may be withdrawn relative to the optical fibre until a second marking is substantially in alignment with the proximal end of the introducer sheath. This enables the distal end of the optical fibre to be set so as to protrude a desired distance beyond the distal end of the introducer sheath in order to administer laser energy. The optical fibre may then be secured to the introducer sheath substantially at the position where the second marking is substantially in alignment with the proximal end of the introducer sheath. Energy from a laser energy source may then be provided to the distal end of the optical fibre. The optical fibre and the introducer sheath can then be withdrawn whilst laser energy is emitted from the distal end of the optical fibre. This has been determined to be a particularly advantageous way of administering laser energy invasively.

It has been determined that it is desirable to deliver laser energy at a rate of approximately 70 J/cm in order to effectively treat veins which may be considered to be of normal size.

However, if an insufficient amount of energy is delivered to the vein wall during the procedure then there is an increased likelihood that the treatment will be ineffective i.e. that the varicose veins will remain after treatment. Also, if during the procedure an excess amount of energy is delivered to the vein then there is a greater risk of the patient experiencing complications as a result of the treatment. For example, the patient may suffer from bruising and paraesthesia.

It is therefore desired to provide an improved medical laser device.

According to an aspect of the present invention there is provided a medical laser device comprising:

a laser source for emitting laser radiation;

an introducer sheath;

an optical fibre which is inserted, in use, within the introducer sheath; and

a withdrawal-assist device which is arranged and adapted to assist a user to withdraw the optical fibre and/or the introducer sheath at a controllable, predetermined or desired rate.

The withdrawal-assist device is preferably arranged to determine either: (i) the speed, velocity or rate at which the optical fibre and/or the introducer sheath are being withdrawn in use; and/or (ii) the speed, velocity or rate at which the optical fibre and/or the introducer sheath should be withdrawn in use.

According to an embodiment the withdrawal-assist device preferably comprises a device which is arranged or located, in use, in close proximity to the introducer sheath and/or the optical fibre.

The withdrawal-assist device may be arranged to contact the introducer sheath and/or the optical fibre. According to this embodiment the withdrawal-assist device may comprise one or more surfaces, wheels or rollers which engage with the introducer sheath and/or the optical fibre.

According to another embodiment the withdrawal-assist device may be arranged to detect movement of the introducer sheath and/or the optical fibre without contacting the introducer sheath and/or the optical fibre. According to this embodiment the withdrawal-assist device may comprise an infra-red (IR), light or electromagnetic wave source and an infra-red, light or electromagnetic wave detector or sensor. The infra-red, light or electromagnetic source preferably emits radiation which is directed, in use, on to the introducer sheath and/or the optical fibre and the infra-red, light or electromagnetic wave detector or sensor preferably detects or senses radiation reflected from the introducer sheath and/or the optical fibre. The light source preferably comprises a laser or Light Emitting Diode (LED) and the optical detector or sensor preferably comprises one or more Complimentary Metal Oxide Semiconductor (CMOS) detectors or sensors and/or one or more Charge Coupled Device (CCD) detectors or sensors.

According to an alternative embodiment the withdrawal-assist device may comprise a device which is arranged or located, in use, at a remote location to the introducer sheath and/or the optical fibre. According to this embodiment the withdrawal-assist device may comprise an optical, camera or vision system which views or observes the introducer sheath and/or the optical fibre.

According to an embodiment the withdrawal-assist device may comprise a first detector means and a second detector means, the second detector means being spaced apart from the first detector means and wherein the withdrawal assist device determines the speed, velocity or rate of withdrawal of the optical fibre and/or the introducer sheath by triangulation. The first and/or second detector means may comprise a laser device or other means for transmitting electromagnetic radiation. According to another embodiment, the optical fibre and/or the introducer sheath may include a transmitter and three receivers may be positioned around the optical fibre and/or the introducer sheath in order to determine the position of the optical fibre and/or the introducer sheath as a function of time by triangulation.

According to an embodiment the withdrawal-assist device may be arranged to contact the optical fibre and/or the introducer sheath in order to determine either: (i) the speed, velocity or rate at which the optical fibre and/or the introducer sheath are being withdrawn in use; and/or (ii) the speed, velocity or rate at which the optical fibre and/or the introducer sheath should be withdrawn in use.

According to an embodiment the withdrawal-assist device may be arranged to determine in a substantially contactless manner with relation to the optical fibre and/or the introducer sheath either: (i) the speed, velocity or rate at which the optical fibre and/or the introducer sheath are being withdrawn in use; and/or (ii) the speed, velocity or rate at which the optical fibre and/or the introducer sheath should be withdrawn in use.

The withdrawal-assist device is preferably arranged to determine mechanically, electro-optically, optically, electrically, electronically, inductively, capacitively, electromagnetically or magnetically either: (i) the speed, velocity or rate at which the optical fibre and/or the introducer sheath are being withdrawn in use; and/or (ii) the speed, velocity or rate at which the optical fibre and/or the introducer sheath should be withdrawn in use.

The optical fibre and/or the introducer sheath preferably comprise one or more markings, graduated markings, sensors, transducers, transmitters, receivers, transponders, coatings, grooves or projections which enable the speed, velocity or rate of withdrawal of the optical fibre and/or the introducer sheath to be determined or estimated by the withdrawal-assist device.

The one or more markings, graduated markings, sensors, transducers, transmitters, receivers, transponders, coatings, grooves or projections may be provided on an inner surface of the optical fibre and/or the introducer sheath.

The one or more markings, graduated markings, sensors, transducers, transmitters, receivers, transponders, coatings, grooves or projections may be provided within or internal to the optical fibre and/or the introducer sheath.

The one or more markings, graduated markings, sensors, transducers, transmitters, receivers, transponders, coatings, grooves or projections may be provided on an outer surface of the optical fibre and/or the introducer sheath.

The optical fibre and/or the introducer sheath is preferably arranged to be received, in use, into or within the withdrawal-assist device. However, other embodiments are contemplated wherein the optical fibre and/or introducer sheath pass over a surface or one or more rollers or wheels.

The withdrawal-assist device may be located in use within a substantially sterile operating field.

A communication link, communication channel or direct or remote connection may be provided between the withdrawal-assist device and the laser source. The withdrawal-assist device may be arranged and adapted in a mode of operation to send and/or receive information or data to and/or from the laser source.

The withdrawal-assist device may be arranged and adapted in a mode of operation to communicate with the laser source in order to adjust the energy, power, repetition rate or another parameter relating to the laser radiation emitted by the laser source and/or delivered by the optical fibre.

According to the preferred embodiment the medical laser device preferably comprises a visual and/or audible and/or tactile signal generating means for generating a signal which is either: (i) indicative of the speed, velocity or rate at which the optical fibre and/or the introducer sheath is being withdrawn in use; and/or (ii) indicative of the speed, velocity or rate at which the optical fibre and/or introducer sheath should be withdrawn in use; and/or (iii) indicative of the speed, velocity or rate at which the optical fibre and/or the introducer sheath are being withdrawn in use should be varied, increased or decreased.

The preferred device may comprise means arranged to adjust the energy, power, repetition rate or another parameter relating to the laser radiation emitted by the laser source and/or delivered by the optical fibre as a function of the position of the optical fibre and/or the introducer sheath within or along a vein.

The preferred device may comprise means arranged to adjust the energy, power, repetition rate or another parameter relating to the laser radiation emitted by the laser source and/or delivered by the optical fibre as a function of vein diameter.

The laser source may be arranged and adapted to be operated in either a pulsed, continuous or quasi-continuous mode of operation.

According to another aspect of the present invention there is provided a method comprising:

inserting an optical fibre within an introducer sheath;

delivering laser radiation to the optical fibre; and

using a withdrawal-assist device to assist a user to withdraw the optical fibre and/or the introducer sheath at a controllable, predetermined or desired rate.

According to another aspect of the present invention there is provided a withdrawal-assist device for assisting a user to withdraw an optical fibre and/or introducer sheath at a controllable, predetermined or desired rate, the device comprising:

a sensor or detector for detecting one or more markings, graduated markings, sensors, transducers, transmitters, receivers, transponders, coatings, grooves or projections on or within an optical fibre and/or on or within an introducer sheath.

The sensor or detector preferably comprises a sensor or detector for detecting a plurality of graduated markings on or within an introducer sheath.

The withdrawal-assist device preferably comprises one or more visual and/or audible and/or tactile signal generating means for generating a signal which instructs a user either of: (i) the speed, velocity or rate at which an optical fibre and/or an introducer sheath is being withdrawn in use; and/or (ii) the speed, velocity or rate at which an optical fibre and/or an introducer sheath should be withdrawn in use; and/or (iii) whether the speed, velocity or rate at which an optical fibre and/or an introducer sheath are being withdrawn in use should be varied, increased or decreased.

According to another aspect of the present invention there is provided a medical laser device comprising:

a laser source for emitting laser radiation;

an introducer sheath;

an optical fibre which is inserted, in use, within the introducer sheath; and

a withdrawal-assist device which is arranged and adapted to assist a user to withdraw the optical fibre and/or the introducer sheath at a controllable, predetermined or desired rate, wherein the withdrawal-assist device is arranged to determine the speed, velocity or rate at which the optical fibre and/or the introducer sheath are being withdrawn and to produce a visual and/or audible and/or tactile signal or indication which instructs a user of either: (i) the speed, velocity or rate at which the optical fibre and/or the introducer sheath are being withdrawn in use; and/or (ii) the speed, velocity or rate at which the optical fibre and/or the introducer sheath should be withdrawn in use; and/or (iii) whether the speed, velocity or rate at which the optical fibre and/or the introducer sheath are being withdrawn in use should be varied, increased or decreased.

According to another aspect of the present invention there is provided a medical laser device comprising:

a laser source for emitting laser radiation;

an introducer sheath;

an optical fibre which is inserted, in use, within the introducer sheath;

a withdrawal-assist device which is arranged and adapted to assist a user to withdraw the optical fibre and/or the introducer sheath at a controllable, predetermined or desired rate, wherein the withdrawal-assist device is arranged to: (i) determine the speed, velocity or rate at which the optical fibre and/or the introducer sheath are being withdrawn; and/or (ii) output a signal to the laser source in order to adjust the energy, power, repetition rate or another parameter relating to the laser radiation being emitted by the laser source and/or being delivered by the optical fibre.

According to another aspect of the present invention there is provided a medical laser device comprising:

a laser source for emitting laser radiation;

an introducer sheath;

an optical fibre which is inserted, in use, within the introducer sheath;

a withdrawal-assist device which is arranged and adapted to assist a user to withdraw the optical fibre and/or the introducer sheath at a controllable, predetermined or desired rate; and

means which are arranged to adjust the energy, power, repetition rate or another parameter relating to the laser radiation being emitted by the laser source and/or being delivered by the optical fibre as a function of the position of the optical fibre and/or the introducer sheath within or along a vein or as a function of vein diameter.

According to another aspect of the present invention there is provided a method of endovenous laser treatment comprising:

introducing an introducer sheath into a blood vessel;

inserting an optical fibre having a distal tip within the introducer sheath;

extending a distal tip of the optical fibre beyond the introducer sheath; and

using a withdrawal-assist device to assist a user to manually withdraw the optical fibre and/or the introducer sheath at a controllable, predetermined or desired rate.

The withdrawal-assist device may comprise one or more rollers, wheels or devices for contacting either the optical fibre and/or the introducer sheath and wherein the withdrawal-assist device determines either: (i) the speed, velocity or rate at which the optical fibre and/or the introducer sheath are being withdrawn in use; and/or (ii) the speed, velocity or rate at which the optical fibre and/or the introducer sheath should be withdrawn in use.

The withdrawal-assist device may comprise a detector for detecting one or more markings or graduations on or within the optical fibre and/or the introducer sheath.

The method preferably further comprises providing a visual and/or audible and/or tactile signal or indication which instructs a user of either: (i) the speed, velocity or rate at which the optical fibre and/or the introducer sheath are being withdrawn in use; and/or (ii) the speed, velocity or rate at which the optical fibre and/or the introducer sheath should be withdrawn in use; and/or (iii) whether the speed, velocity or rate at which the optical fibre and/or the introducer sheath are being withdrawn in use should be varied, increased or decreased.

The withdrawal-assist device is preferably arranged to communicate with a laser source in order to automatically adjust the energy, power, repetition rate or another parameter relating to the laser radiation being emitted by the laser source and/or being delivered by the optical fibre.

According to another aspect of the present invention there is provided a medical laser device comprising:

a laser source for emitting laser radiation;

an optical fibre, the optical fibre comprising one or more markings, graduated markings, sensors, transducers, transmitters, receivers, transponders, coatings, grooves or projections; and

a withdrawal-assist device which is arranged and adapted to assist a user to withdraw the optical fibre at a controllable, predetermined or desired rate, wherein the withdrawal-assist device determines or estimates, in use, the speed, velocity or rate of withdrawal of the optical fibre.

According to another aspect of the present invention there is provided a method comprising:

providing a laser source for emitting laser radiation and an optical fibre, the optical fibre comprising one or more markings, graduated markings, sensors, transducers, transmitters, receivers, transponders, coatings, grooves or projections; and

using a withdrawal-assist device to assist a user to withdraw the optical fibre at a controllable, predetermined or desired rate, wherein the withdrawal-assist device determines or estimates the speed, velocity or rate of withdrawal of the optical fibre.

The preferred embodiment relates to an improved medical laser device which allows an operator to adjust the energy delivered to the walls of the patient's vein whilst the treatment is progressing.

Various embodiments of the present invention will now be described, by way of example only, and with reference to the accompanying drawings in which:

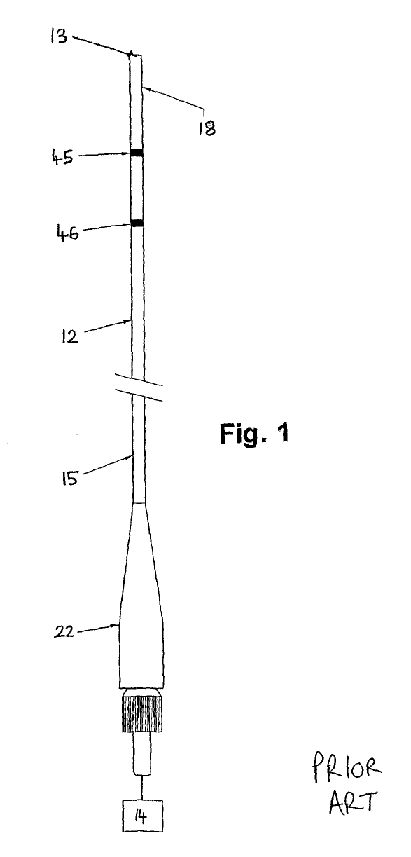

FIG. 1 shows a known medical laser device;

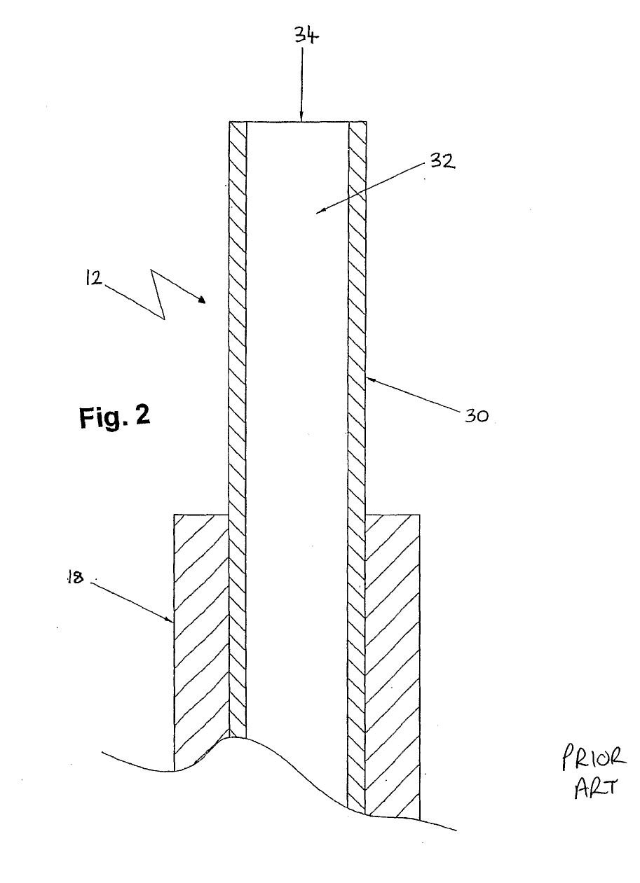

FIG. 2 shows in greater detail the distal end section of the optical fibre shown FIG. 1;

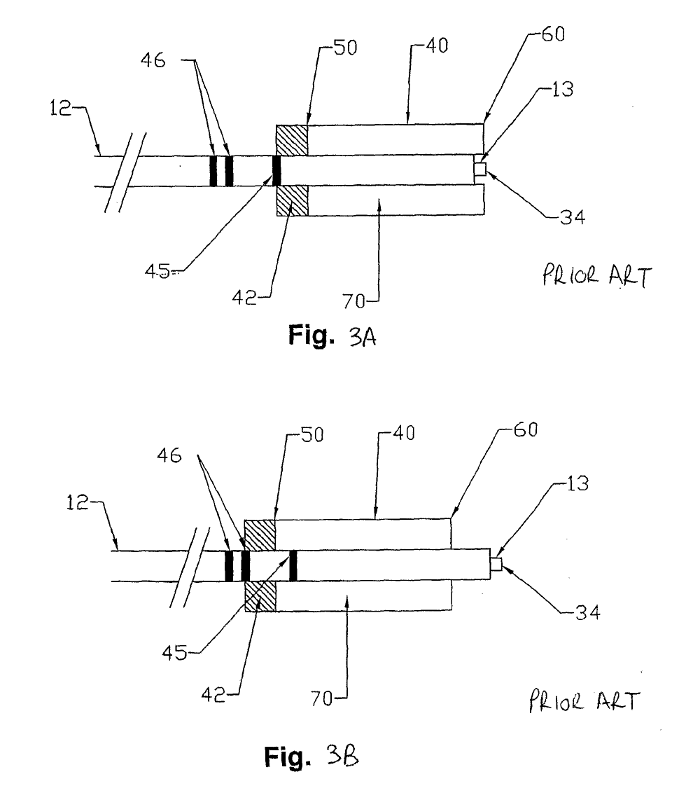

FIG. 3A shows an optical fibre having been initially inserted into an introducer sheath and FIG. 3B shows the introducer sheath having been subsequently withdrawn relative to the optical fibre; and

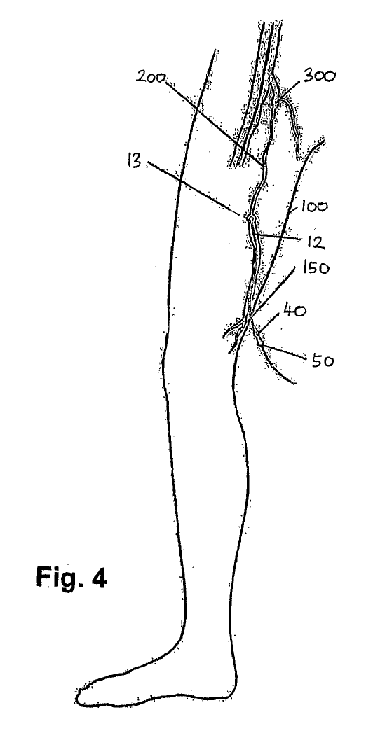

FIG. 4 shows an optical fibre and introducer sheath inserted into a vein.

A known medical laser device is shown in FIGS. 1 and 2 and comprises an optical fibre 12 having a distal end 13 and a proximal end 15. The optical fibre is coupled to a laser energy source 14 via a connector 22. The optical fibre 12 has a length of 2.5 m±0.1 m and is provided with a protective buffer layer 18. Two markings 45,46 are arranged on the optical fibre 12 at a predetermined distance from distal end 13. The markings 45,46 are provided around substantially the whole circumference of the protective buffer layer 18 of the optical fibre 12. The laser energy source 14 comprises an 810 nm diode laser manufactured by DIOMED Ltd., United Kingdom. The connector 22 may comprise any suitable connector/fibre terminator such as a standard sub-miniature A (SMA) connector (as shown) or other proprietary connector.

The distal end 13 of the optical fibre 12 is shown in more detail in FIG. 2. The optical fibre 12 may be arranged so as to be capable of withstanding environmental temperatures of −10° C. to 120° C. The optical fibre 12 may comprise a glass core 32 with a fibre tip 34, a cladding layer 30 surrounding the core 32, and an outer protective buffer layer 18.

The core 32 has a higher refractive index than the cladding 30 and as a result laser energy is guided along the core 32 by Total Internal Reflection. The optical fibre 12 has a 600 μm diameter glass core, for example CF01493-14 available from OFS, USA. The buffer layer 18 is partially stripped back a short distance (e.g. approx. 1 cm) from the fibre tip 34 so that the diameter of the optical fibre 12 in the region around the fibre tip 34 is approximately 0.6 mm.

For efficient operation of the device the surface quality of the fibre tip 34 is high. The fibre tip 34 is substantially free from defects within a central aperture of 88% of the diameter of the fibre optic core 32 with all sharp edges having been previously removed from the fibre tip 34. Other defects (if present) do not scatter light outside a 0.37 numerical aperture (NA), and do not cause localised heating when illuminated with 60 W of laser light at 810 nm evenly distributed over a 0.37 NA. The optical fibre 12 is capable of withstanding a force of 2 kg, applied between the connector/terminator (SMA) 22, and the distal end 13, without damage.

The relationship between the position of the markings or indicators 45,46 and an introducer sheath 40 or the like will now be described in more detail with reference to FIGS. 3A and 3B.

FIG. 3A shows the optical fibre 12 having been initially inserted within an introducer sheath 40 so that the distal end 13 of the optical fibre 12 is substantially flush or otherwise aligned with the distal end 60 of the introducer sheath 40. Marking 45 is shown substantially aligned with the proximal end 50 of the introducer sheath 40. The introducer sheath 40 is then withdrawn relative to the optical fibre 12 until marking 46 is substantially aligned with the proximal end 50 of the introducer sheath 40. As shown in FIG. 3B, the distal end 13 of the optical fibre 12 then protrudes beyond the distal end 60 of the introducer sheath 40 by a predetermined amount, e.g. approx. 3 cm.

The introducer sheath 40 comprises a hollow tube 70 with a friction seal 42 at the proximal end 50 to prevent blood loss during insertion into blood vessels. The placement of the introducer sheath 40 through skin and tissue provides access into an area to be treated and acts as a guide for introducing instrumentation and other apparatus. The introducer sheath 40 may comprise a five French introducer sheath as provided in ELVT (RTM) kits (EVLT/PK-02, EVLT/PK-02-EL, EVLT/PPK, EVLT/70-SL) available from DIOMED Inc., USA.

Markings 45,46 are provided around the whole circumference of the protective buffer layer 18 of the optical fibre 12, and are provided at predetermined distances from the distal end 13 of the optical fibre 12. The predetermined distances are arranged to correspond to predetermined positions of the distal end 13 of the optical fibre 12 relative to the distal end 60 of the introducer 40. The predetermined positions represent both alignment of the respective distal ends 13,60 as shown in FIG. 3A and also protrusion of the distal end 13 of the optical fibre 12 beyond the distal end 60 of the introducer 40 as shown in FIG. 3B.

The location of the first marker 45 corresponds to a depth of insertion of the optical fibre 12 into the introducer sheath 40 such that the distal end 13 of the optical fibre 12 (i.e. fibre tip 34) is very closely aligned or is substantially flush with the distal end 60 of the introducer sheath 40. The introducer sheath 40 may be, for example, 35 cm or 45 cm long and may provided with a 2.1 cm friction seal 42 making a total length 37.1 cm or 47.1 cm. Accordingly, the first marking 45 is provided at 371 mm±1 mm or 471 mm±1 mm from the distal end 13 of the optical fibre 12. Alternatively, the friction seal may be fixed to the optical fibre.

The location of the second marking 46 corresponds to a position where the distal end 13 of the optical fibre 12 extends or projects 30 mm±1 mm beyond the distal end 60 of the introducer sheath 40. Accordingly, the second marking is provided at 401 mm±1 mm or 501 mm±1 mm from the distal end 13 of the optical fibre 12.

The known medical laser device is preferably used for Endovenous Laser Treatment (“EVLT”) (RTM) of, for example, a human leg.

In order to treat a human leg 100 local anaesthesia such as 0.3% or otherwise dilute lidocaine is preferably administered perivenously along the Greater Saphenous Vein (GSV) 200 (see FIG. 4). Ultrasound guidance may be used. Percutaneous entry into the Greater Saphenous Vein 200 may be made with a needle at a point 150 which is preferably 25-45 cm below the Saphenofemoral Junction (SFJ) 300. A 0.035″ (0.089 cm) J-tip guide wire may be passed into the Greater Saphenous Vein 200 and a five French introducer sheath 40 may be passed over the guide wire up to the Saphenofemoral Junction 300.

The guide wire is preferably removed and a sterile 600 nm diameter core, bare-tipped optical fibre 12 is preferably introduced into the vein 200 through the introducer sheath 40 until the first marking 45 is preferably aligned with the proximal end 50 of the introducer sheath 40 (which corresponds with the portion of the friction seal furthermost from distal end 60). The optical fibre 12 and the proximal end 50 of the introducer sheath 40 are then preferably fixed or held together. They are then preferably moved together and positioned using ultrasound guidance at a location which is preferably about 1-2 cm below the Saphenofemoral Junction 300 within the Greater Saphenous Vein 200. Once positioned, the introducer sheath 40 is then preferably released from the optical fibre 12 and the introducer sheath 40 is preferably withdrawn relative to the optical fibre 12 until the proximal end 50 of the introducer sheath 40 is preferably aligned with the second marking 46 on the optical fibre 12. When the introducer sheath 40 and the optical fibre 12 are in this position then the distal end 13 of the optical fibre 12 will preferably extend approximately 30 mm beyond the distal end 60 of the introducer sheath 40. This position has been found to be an effective position for administering laser energy in EVLT (RTM) treatment. A minimum extension of 20 mm of the optical fibre 12 beyond the end of the introducer sheath 40 may be considered as being a suitable distance for administering laser energy in EVLT (RTM) treatment without causing thermal damage to the introducer sheath 40. The optical fibre 12 and the proximal end 50 of the introducer sheath 40 are then preferably secured together with non-permanent means. Pulsed 810 nm wavelength laser radiation from a laser energy source (not shown) is preferably administered with a power of 10-12 Watts, in, for example, pulses of 0.8-1.0 second duration at 1 second pulse intervals to treat the Greater Saphenous Vein 200. The introducer sheath 40 together with the optical fibre 12 are then slowly withdrawn in preferably 2-5 mm increments whilst administering the laser radiation. Manual compression may be applied over a red aiming beam in order to achieve vein wall apposition around the laser fibre tip 34. According to the known approach the clinician is instructed to withdraw the introducer sheath 2-3 mm between 1 second laser pulses. This process is continued until the desired length of the vein has been treated.

The preferred embodiment relates to an improvement to the known medical laser device and comprises a withdrawal-assist device.

According to the preferred embodiment the laser is preferably arranged to provide a continuous rather than a pulsed output. Graduated markings are preferably provided on the external surface of the introducer sheath 40. The graduated markings preferably enable the introducer sheath 40 to be withdrawn at a prescribed or desired rate. The graduated markings may also indicate how far the introducer sheath 40 has been withdrawn from a patient's body. A visible and/or audible and/or tactile indicator may be provided in order to assist an operator or surgeon to remove or withdraw the introducer sheath 40 and optical fibre 12 at a controlled speed.

According to an embodiment the visible and/or audible and/or tactile indicator may form part of the laser system coupled to the optical fibre 12. For example, if graduations are located on the external surface of the introducer sheath 40 at 1 cm intervals then the audible and/or visual and/or tactile indicator may be arranged so as to be activated once per second. A user is therefore assisted in withdrawing the introducer sheath 40 and optical fibre 12 at a controlled or desired speed or rate of 1 cm/sec. It will be apparent that a user should ensure that a new graduation on the introducer sheath 40 was revealed every time the indicator bleeped and/or flashed and/or vibrated.

The preferred embodiment is particularly advantageous in that it enables bio feedback to be provided in order to control the rate at which the introducer sheath 40 is withdrawn. The continuous withdrawal of the introducer sheath 40 also results in a more uniform illumination of the internal surface of the blood vessel. In contrast, the known medical device comprises a pulsed laser source and the introducer sheath 40 and optical fibre 12 are withdrawn in a stepped manner. This may, in some circumstances, cause relatively high intensity regions of illumination at, for example, 2-3 mm intervals along the length of the blood vessel. The medical laser device according to the preferred embodiment avoids any such potential problem by preferably using a continuous laser source, providing graduations along the length of the introducer sheath 40 and using a withdrawal-assist device to enable the user to withdraw the introducer sheath 40 in a controlled manner or at a controlled rate. The introducer sheath and/or optical fibre are preferably withdrawn manually although less preferred embodiments are contemplated wherein the introducer sheath and/or optical fibre are withdrawn automatically or semi-automatically.

According to another embodiment a visual and/or audible and/or tactile indicator may be used in conjunction with a pulsed laser source as an aid to withdrawing the introducer sheath 40. This allows a hybrid approach wherein the optical fibre 12 is withdrawn a longer distance e.g. 1-3 cm during a longer pulse-length. According to this embodiment, the laser source is operated in a quasi-continuous mode.

According to the preferred embodiment the sheath 40 and the optical fibre 12 are preferably arranged to be withdrawn at a controlled rate so as to ensure that the required amount of energy is delivered to the treatment site in a controlled manner.

The energy delivered to the vein wall during the procedure is preferably determined by two factors namely the laser power emitted at the distal end 13 of the optical fibre 12 and the rate at which the optical fibre 12 is withdrawn from the vein 200.

According to the preferred embodiment the operator is preferably able to achieve the desired delivered energy by adjusting the withdrawal rate of the optical fibre 12 whilst the treatment is progressing. However, according to another embodiment the operator may alternatively and/or additionally adjust the laser power emitted from by the laser source and/or delivered from the distal end 13 of the optical fibre 12 whilst the optical fibre 12 is being withdrawn.

The introducer sheath 40 which surrounds the optical fibre 12 may according to one embodiment be arranged to pass through a slot or cavity in a withdrawal-assist device which aids the operator to control the withdrawal rate of the introducer sheath 40. The slot or cavity in the withdrawal-assist device may be equipped with a means to determine and/or control the speed at which the introducer sheath 40 is being withdrawn.

According to another embodiment the withdrawal-assist device may comprise a surface or one or more rollers or wheels with engage with the optical fibre 12 and/or the introducer sheath 40. Measurement of the speed of rotation of the rollers or wheels can be used to indicate the rate at which the optical fibre 12 and/or introducer sheath are being withdrawn.

Various different means may be used in order to determine and/or control the withdrawal speed of the introducer sheath 40. The particular withdrawal-assist device used may depend upon the required sensitivity of the device, the required reliability of the device and the relative expense of the device.

In one embodiment, the means for determining and/or controlling the desired withdrawal speed of the introducer sheath 40 may comprise one or more roller wheels or belts. Graduations are preferably provided on the external surface of the introducer sheath 40. One or more transducers such as a magnetic transducer or Hall effect transducer may be provided within the slot or cavity of the withdrawal-assist device in order to sense the graduations preferably provided on the outer surface of the introducer sheath 40. According to less preferred embodiments the graduations may be provided on an inner surface of the introducer sheath 40 or on the optical fibre 12.

In operation, the withdrawal-assist device may be placed within a sterile operating field. The withdrawal-assist device may preferably be arranged so as to be capable of withstanding re-sterilisation and therefore can be used in multiple procedures.

Alternatively, the withdrawal-assist device may be located outside of a sterile operating field. According to this embodiment it is not necessary for the withdrawal-assist device to be a sterile device, but merely that the part of the introducer sheath 40 which is in contact with the sterile operating field is protected. Such protection may be provided, for example, by isolating the introducer sheath 40 from the withdrawal-assist device with a sterile shroud.

According to an embodiment at least part of the withdrawal-assist device may comprise a disposable single-use sterile device.

Prior to the start of the preferred procedure, the operator preferably selects the desired energy E to be delivered. The desired energy E to be delivered may be set depending upon the particular diameter of the vein 200 to be treated.

The withdrawal-assist device may be arranged to communicate with the laser source. For example, a serial interface link may be provided so that the actual energy E′ delivered during the procedure can be determined. The laser device is preferably provided with a means for comparing the actual energy E′ delivered during the procedure to the energy E which was desired to be delivered. A means for generating a feedback signal is preferably provided. The feedback signal is preferably arranged so as to be indicative of the difference between the desired energy E and the actual delivered energy E′.

The feedback signal may be communicated to the operator, by, for example, means of a visual and/or audible and/or tactile signal. The operator can then adjust the rate of withdrawal of the introducer sheath 40 so that the desired amount of energy is actually delivered to the vein.

According to an alternative embodiment the feedback signal may be provided to the laser source 14 so that the power or energy emitted from the laser source 14 or otherwise delivered to the patient from the distal end of the optical fibre 12 may be manually or automatically adjusted so that the desired amount of energy or laser power is delivered to the vein.

The energy which is delivered by the preferred medical laser device may be arranged so as to vary if or as the diameter of the vein to be treated varies along the length of the vein. For example, it may be desired that the energy delivered at one location of the vein 200 is greater than the energy which should be delivered at another location further along the vein 200. In order to take account of this variation in vein diameter as a function of position along the length of the vein, ultrasound scans of the vein 200 may be performed prior to treatment. The pre-treatment ultrasound scans may then be used to accurately determine the dimensions of the vein along the length of the vein to be treated. The vein dimensions as determined from these scans may then be communicated to the withdrawal-assist device during the procedure or may be stored within the withdrawal-assist device so that the energy which is desired to be delivered to the vein can be manually or automatically varied as the introducer sheath 40 and/or optical fibre 12 are withdrawn along the length of the vein 200. The operator can therefore adjust the withdrawal rate of the introducer sheath 40 accordingly so that the correct amount of energy is preferably delivered to each part of the vein.

According to an embodiment an infra-red imaging system may be used to view an area of a patient's body or an area of tissue. According to this embodiment near surface veins may be detected and images of the veins may be projected onto the skin surface or the body of the patient. The imaging system may also be used to image the withdrawal of the introducer sheath and/or the optical fibre.

According to an embodiment a camera or other optical system may be used to image or view markings on the introducer sheath and/or optical fibre. The camera or optical system may determine the rate of withdrawal of the introducer sheath and/or the optical fibre. The camera or optical system may determine the rate of withdrawal of the introducer sheath and/or the optical fibre by observing graduations or other markings on the introducer sheath and/or the optical fibre.

According to an embodiment an optical system may be provided which comprises an electromagnetic radiation source and one or more detectors. For example, the system may comprise a LED transmitter and one or more photodetectors. The introducer sheath and/or the optical fibre may be withdrawn so that electromagnetic radiation from the LED passes across the introducer sheath and/or the optical fibre which is being withdrawn. The one or more photodetectors can then determine the rate of withdrawal of the introducer sheath and/or the optical fibre. The optical system may, according to one embodiment, comprise a system similar to that provided in an optical mouse and the introducer sheath and/or optical fibre may be pulled through the system which detects the rate of withdrawal of the introducer sheath and/or optical fibre.

A yet further embodiment is contemplated wherein the speed, velocity or rate of withdrawal of the optical fibre and/or introducer sheath are determined by triangulation. According to this embodiment a first detector means and a second detector means are provided separated by a known distance. The first detector means observes the optical fibre and/or the introducer sheath and determines the angle between the optical fibre and/or the introducer sheath, the first detector means and the second detector means. Similarly, the second detector means observes the optical fibre and/or the introducer sheath and determines the angle between the optical fibre and/or the introducer sheath, the second detector means and the first detector means. Using the principle of triangulation, the position and/or relative distance from a fixed point of the optical fibre and/or the introducer sheath can be determined at any instant in time. According to this embodiment repeated observations may be made allowing the position and/or relative distance to be determined as a function of time and hence enabling the speed, velocity or rate of withdrawal of the optical fibre and/or introducer sheath to be determined. According to another embodiment, the optical fibre and/or the introducer sheath may include a transmitter and three receivers may be positioned around the optical fibre and/or the introducer sheath in order to determine the position of the optical fibre and/or the introducer sheath as a function of time by triangulation.

Although the present invention has been described with reference to preferred embodiments, it will be understood by those skilled in the art that various changes in form and detail may be made to the particular embodiments discussed above without departing from the scope of the invention as set forth in the accompanying claims.

1-42. (canceled)

43. A medical laser device comprising:
a laser source for emitting laser radiation;
an introducer sheath;
an optical fiber which is inserted, in use, within said introducer sheath; and
a withdrawal-assist device which is arranged and adapted to assist a user to withdraw said optical fiber and/or said introducer sheath at a desired rate, wherein the withdrawal-assist device includes an engaging element which engages said optical fiber and/or said introducer sheath for measuring the withdrawal rate of said optical fiber and/or said introducer sheath.

44. A medical laser device as recited in claim 43, wherein said engaging element has one or more wheels or rollers.

45. A medical laser device as recited in claim 43, wherein said withdrawal-assist device measures the withdrawal rate by measuring the rate of rotation of the engaging element.

46. A medical laser device as recited in claim 44, wherein said withdrawal-assist device measures the withdrawal rate by measuring the rate of rotation of the wheels or rollers.

47. A medical laser device as recited in claim 43, wherein said optical fiber and/or said introducer sheath further comprise one or more markings, graduated markings, sensors, transducers, transmitters, receivers, transponders, coatings, grooves or projections which enable the rate of withdrawal of said optical fiber and/or said introducer sheath to be determined by said withdrawal-assist device.

48. A medical laser device as recited in claim 43, wherein said optical fiber and/or said introducer sheath is arranged to be received, in use, into or within said withdrawal-assist device.

49. A medical laser device as recited in claim 43, further comprising a communication link, communication channel or direct or remote connection between said withdrawal-assist device and said laser source and wherein said withdrawal-assist device is arranged and adapted in a mode of operation to send and/or receive information or data to and/or from said laser source.

50. A medical laser device as recited in claim 49, wherein said withdrawal-assist device is arranged and adapted in a mode of operation to communicate with said laser source in order to adjust the energy, power, repetition rate or another parameter relating to the laser radiation emitted by said laser source and/or delivered by said optical fiber.

51. A medical laser device as recited in claim 43, further comprising a visual and/or audible and/or tactile signal generator for generating a signal which is indicative of the rate at which said optical fiber and/or said introducer sheath is being withdrawn in use.

52. A medical laser device as recited in claim 43, further comprising a visual and/or audible and/or tactile signal generator for generating a signal which is indicative of the rate at which said optical fiber and/or introducer sheath should be withdrawn in use.

53. A medical laser device as recited in claim 43, further comprising a visual and/or audible and/or tactile signal generator for generating a signal which is indicative of the rate at which said optical fiber and/or said introducer sheath is being withdrawn should be varied.

54. A medical laser device as recited in claim 43, further comprising a visual and/or audible and/or tactile signal generator for generating a feedback signal which is indicative of the difference between a predetermined desired energy E to be delivered to a vein and an actual delivered energy E′ that is delivered to the vein.

55. A medical laser device as recited in claim 43, wherein said optical fiber and/or said introducer sheath are withdrawn automatically or semi-automatically.

56. A medical laser device comprising:
a laser source for emitting laser radiation;
an introducer sheath;
an optical fiber which is inserted, in use, within said introducer sheath; and
a withdrawal-assist device which is arranged and adapted to assist a user to withdraw said optical fiber and/or said introducer sheath at a desired rate, wherein the withdrawal-assist device includes one or both of the following for measuring the withdrawal rate of said optical fiber and/or said introducer sheath:
an optical detector including a complementary metal oxide semiconductor (CMOS) detector or charge coupled device (CCD) detector; or
a triangulation element having a first detector means and a second detector means, said second detector means being spaced apart from said first detector means.

57. A medical laser device as recited in claim 56, wherein said optical fiber and/or said introducer sheath further comprise one or more markings, graduated markings, sensors, transducers, transmitters, receivers, transponders, coatings, grooves or projections which enable the rate of withdrawal of said optical fiber and/or said introducer sheath to be determined by said withdrawal-assist device.

58. A medical laser device as recited in claim 56, wherein said optical fiber and/or said introducer sheath is arranged to be received, in use, into or within said withdrawal-assist device.

59. A medical laser device as recited in claim 56, further comprising a communication link, communication channel or direct or remote connection between said withdrawal-assist device and said laser source and wherein said withdrawal-assist device is arranged and adapted in a mode of operation to send and/or receive information or data to and/or from said laser source.

60. A medical laser device as recited in claim 59, wherein said withdrawal-assist device is arranged and adapted in a mode of operation to communicate with said laser source in order to adjust the energy, power, repetition rate or another parameter relating to the laser radiation emitted by said laser source and/or delivered by said optical fiber.

61. A medical laser device as recited in claim 56, further comprising a visual and/or audible and/or tactile signal generator for generating a signal which is either: (i) indicative of the rate at which said optical fiber and/or said introducer sheath is being withdrawn in use; and/or (ii) indicative of the rate at which said optical fiber and/or introducer sheath should be withdrawn in use; and/or (iii) indicative of the rate at which said optical fiber and/or said introducer sheath are being withdrawn in use should be varied, increased or decreased.

62. A medical laser device as recited in claim 56, further comprising a visual and/or audible and/or tactile signal generator for generating a feedback signal which is indicative of the difference between a predetermined desired energy E to be delivered to a vein and an actual delivered energy E′ that is delivered to the vein.

63. A medical laser device as recited in claim 56, wherein said optical fiber and/or said introducer sheath are withdrawn automatically or semi-automatically.

64. A method comprising:
inserting an optical fiber within an introducer sheath;
delivering laser radiation to said optical fiber; and
using a withdrawal-assist device to assist a user to withdraw said optical fiber and/or said introducer sheath at a desired rate, wherein the withdrawal-assist device includes an engaging element which engages said optical fiber and/or said introducer sheath for measuring the withdrawal rate of said optical fiber and/or said introducer sheath.

65. A method as recited in claim 64, further comprising providing a visual and/or audible and/or tactile signal or indication which informs a user of either: (i) the speed, velocity or rate at which said optical fiber and/or said introducer sheath are being withdrawn in use; and/or (ii) the speed, velocity or rate at which said optical fiber and/or said introducer sheath should be withdrawn in use; and/or (iii) whether the speed, velocity or rate at which said optical fiber and/or said introducer sheath are being withdrawn in use should be varied, increased or decreased.

66. A method as recited in claim 64, wherein said withdrawal-assist device communicates with a laser source in order to automatically adjust the energy, power, repetition rate or another parameter relating to the laser radiation being emitted by said laser source and/or being delivered by said optical fiber.
